(19) United States
(12) Patent Application Publication
(10) Pub. No.: US 2022/0096810 A1
(43) Pub. Date: Mar. 31, 2022

(54) CONNECTOR WITH SEALING PORTION

(71) Applicant: CareFusion 303, Inc., San Diego, CA (US)

(72) Inventor: Choi Ting Lau, Santee, CA (US)

(21) Appl. No.: 17/035,131

(22) Filed: Sep. 28, 2020

(51) Int. Cl. A61M 39/10; A61M 39/08

(52) U.S. Cl. CPC A61M 39/10; A61M 39/12; A61M 2205/0216; A61M 39/08

(57) ABSTRACT

Connectors are described herein. A connector includes a coupling portion, a tubing portion, and a sealing portion. The coupling portion is configured to engage with a mating connector. The tubing portion extends from the coupling portion. The tubing portion can include a tubing portion elastic modulus and define a lumen in fluid communication with the coupling portion. The sealing portion can be coupled to the tubing portion. The sealing portion can have a sealing portion elastic modulus, wherein the sealing portion elastic modulus is lower than the tubing portion elastic modulus. The sealing portion is can be configured to sealingly engage against the tubing.

### FIELD OF THE INVENTION

The present disclosure generally relates to connectors, and, in particular, to tubing connectors.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. IV sets can include various components, such as needles/catheters, an IV pump, drip chambers, check valves, access ports, luers, and/or clamps for controlling the fluid flow in the IV line. As can be appreciated, each component of the IV set can be connected to each other via a joint or junction.

In some applications, variations between tubing and connectors can prevent sealing and/or mating between joints or junctions.

### SUMMARY

The disclosed subject matter relates to connectors. In certain embodiments, a connector is disclosed that comprises a coupling portion configured to engage with a mating connector; a tubing portion extending from the coupling portion, the tubing portion comprising a tubing portion elastic modulus and the tubing portion defining a lumen in fluid communication with the coupling portion, wherein the tubing portion is configured to engage with a tubing; and a sealing portion coupled to the tubing portion, the sealing portion comprising a sealing portion elastic modulus, wherein the sealing portion elastic modulus is lower than the tubing portion elastic modulus, and the sealing portion is configured to sealingly engage against the tubing.

In certain embodiments, a method is disclosed that comprises advancing a tubing toward a connector; engaging the tubing with a tubing portion of the connector; and sealingly engaging the tubing with the tubing portion of the connector via a sealing portion coupled to the tubing portion, wherein a sealing portion elastic modulus is lower than a tubing portion elastic modulus.

In certain embodiments, a connector assembly is disclosed that comprises a connector comprising: a coupling portion configured to engage with a mating connector; a tubing portion extending from the coupling portion, the tubing portion comprising a tubing portion elastic modulus and the tubing portion defining a connector lumen in fluid communication with the coupling portion; and a sealing portion coupled to the tubing portion, the sealing portion comprising a sealing portion elastic modulus, wherein the sealing portion elastic modulus is lower than the tubing portion elastic modulus; and a tubing defining a tubing lumen, wherein the tubing is sealingly engaged with the sealing portion and the tubing lumen is in fluid communication with the connector lumen.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates a patient receiving an infusion of a medical fluid through an IV pump according to certain aspects of the present disclosure.

FIG. 2 illustrates a perspective view of a connector, according to certain aspects of the present disclosure.

FIG. 3 illustrates a partial cross-sectional view of the connector of FIG. 2.

FIG. 4 illustrates a perspective view of a connector, according to certain aspects of the present disclosure.

FIG. 5 illustrates a perspective view of a connector with the tubing shown in hidden lines.

FIG. 6 illustrates a perspective view of a connector, according to certain aspects of the present disclosure.

### DETAILED DESCRIPTION

The disclosed connector incorporates a sealing portion coupled to the tubing portion. The sealing portion can have a lower elastic modulus than the connector body, allowing the sealing portion to readily adapt or conform to any imperfections or voids in the connector body and/or the tubing. By adapting to the imperfections or voids of the connector body and/or the tubing, the connector can allow for a secure, leak-free connection, with less manufacturing complexity, and less testing.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to the connection of medical fittings for the administration of medical fluid using the disclosed connector, it is to be understood that this description is only an example of usage and does not limit the scope of the claims. Various aspects of the disclosed connector may be used in any application where it is desirable to provide a secure connection with reduced manufacturing and testing requirements.

The disclosed connector overcomes several challenges discovered with respect to certain conventional connectors. One challenge with certain conventional connectors is that certain conventional connectors require a precision fit between mating surfaces and/or the use of solvent to allow for a secure connection therebetween, leading to connections that may leak and/or separate. Another challenge with certain conventional connectors is that certain conventional connectors may employ an application or size specific design, such as multiple flaring or a taper tip. Often such variations require unique manufacturing tooling and testing, leading to a complex joint portfolio. For example, certain convention pump sets may require the use of more than 12 unique connectors. Because leaking or separated connectors can interrupt the administration of medical fluids and because manufacturing a large portfolio of connectors can lead to complexities in testing and manufacturing, the use of certain conventional connectors is undesirable.

Therefore, in accordance with the present disclosure, it is advantageous to provide a connector as described herein that allows imperfections between mating surfaces to permit secure and leak-free connections. Further, it is advantageous to provide a connector as described herein that can be used in a variety of applications, reducing the number of connectors that need to be manufactured and tested.

Examples of connectors that allow secure and leak-free connections are now described.

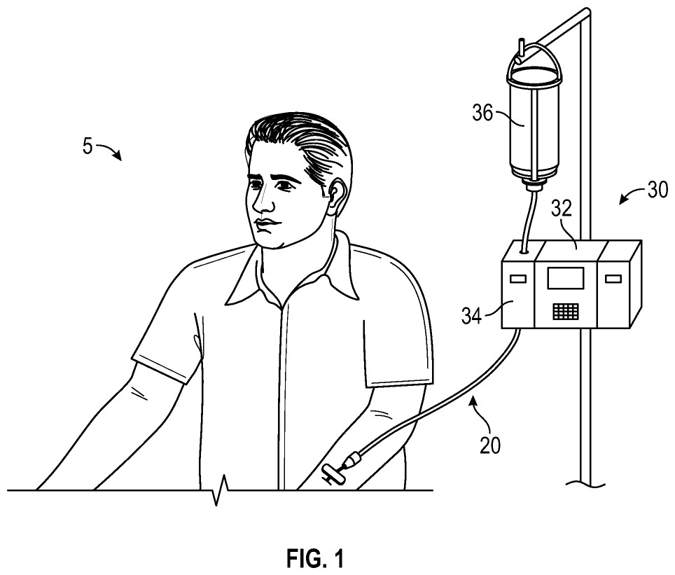

FIG. 1 illustrates a patient 5 receiving an infusion of a medical fluid through an IV pump 30 according to certain aspects of the present disclosure. The IV pump 30 comprises a controller 32 and two pump modules 34. An IV set 20 is connected between a container 36 of the medical fluid and the patient 10. As can be appreciated, the IV set 20 can include various components, such as needles/catheters, drip chambers, check valves, access ports, luers, and/or clamps for controlling the fluid flow in the IV line. As described herein, each component of the IV set can be connected to each other via a connector.

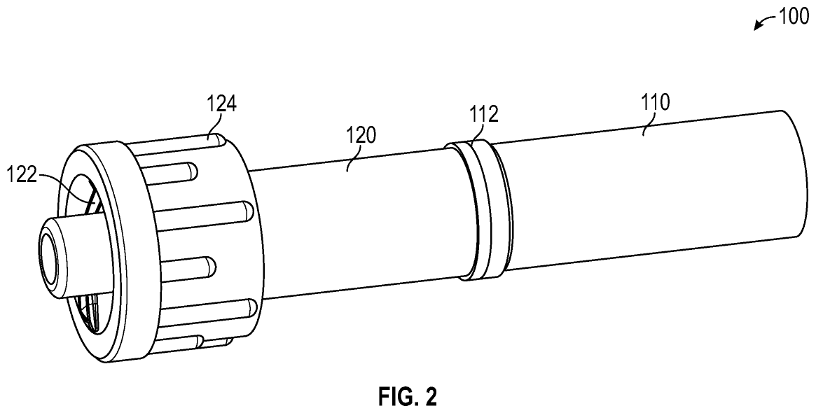

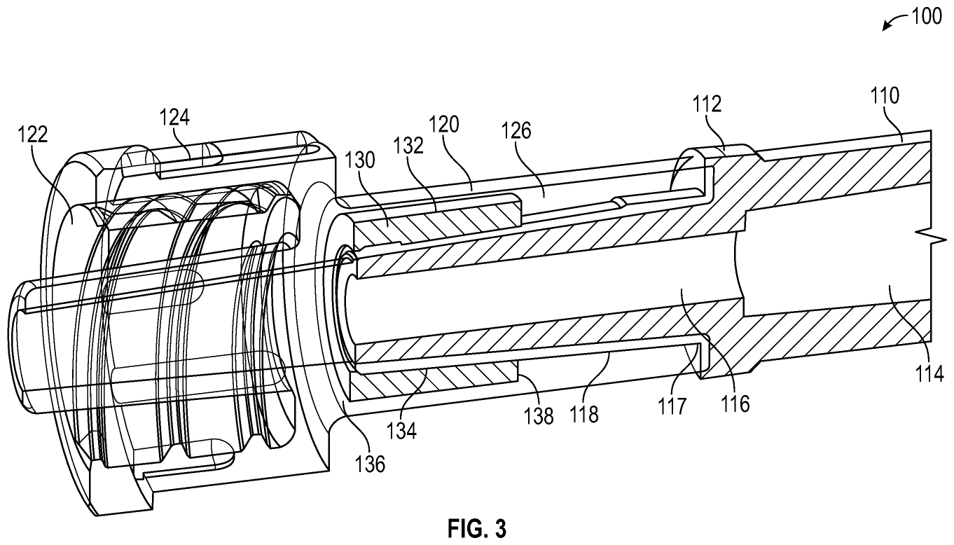

FIG. 2 illustrates a perspective view of a connector 100, according to certain aspects of the present disclosure. FIG. 3 illustrates a partial cross-sectional view of the connector 100 of FIG. 2. In the depicted example, tubing 110 can be terminated with a connector 100 to allow for the connection and/or disconnection of the tubing 110 to allow fluid flow between the tubing 110 and components of the IV set.

As illustrated, the tubing 110 can be coupled with, or engage with a tubing portion 120 of the connector 100. The tubing portion 120 can be in fluid communication with a tubing lumen 114 of the tubing 110 to allow fluid to pass through the connector 100. In some embodiments, an inner surface 117 of the tubing portion 120 defines a connector lumen 126. Optionally, portions of the tubing 110 can be inserted into the connector lumen 126 to engage with, or otherwise couple with the connector 100. The connector lumen 126 can define a bond pocket between the connector 100 and the tubing 110.

Optionally, the tubing 110 can be terminated with a rigid connector 118. As illustrated, the rigid connector 118 of the tubing 110 can be inserted into the connector lumen 126 to couple the tubing 110 with the connector 100. The rigid connector 118 can have a generally tapered construction. As can be appreciated, the rigid connector 118 can have various unique shapes configured to connect with one or more other components. The rigid connector 118 can be formed from a rigid material with a relatively high elastic modulus. In some embodiments, the tubing 110 can include or define a tubing end or collar 112 adjacent to the rigid connector 118 to control the insertion of the tubing 110 into the connector 100.

Fluid can flow from through the connector 100 via a coupling portion 122 extending from the tubing portion 120. The connector lumen 126 can be in fluid communication with the coupling portion 122. In some embodiments, the coupling portion 122 can include a threaded luer connection to facilitate coupling with other IV set components.

Optionally, the coupling portion 122 can include raised features 124 disposed on the surface of the connector 100 to allow a clinician to more easily handle or manipulate the connector 100. The raised features 124 can allow the connector 100 to be rotated to connect or disconnect the connector from other IV set components. Some embodiments of the connector 100 can provide connectors that are compatible with connectors of other portions of fluid delivery systems.

In some embodiments, the connector 100 can have a generally rigid construction. The connector 100 can be formed from materials such as plastic, ceramic, metal, and/or composite. In some embodiments, the connector 100 can be formed from materials that have a generally high elastic modulus.

In the depicted example, the connector 100 can include a sealing portion 130 to sealingly engage with the tubing 110 and/or the rigid connector 118 to securely couple the tubing 110 to the connector 100. Advantageously, the sealing portion 130 can adapt to variations in geometry of the tubing 110 and/or the rigid connector 118, allowing leak-free, secure connections between the tubing 110 and the connector 100 for a wide range of tubing 110 and/or rigid connector 118 geometries.

As illustrated, the sealing portion 130 can conform to the shape of the tubing 110 and/or the rigid connector 118 to fill any gaps, voids, or imperfections between the mating surfaces of the tubing portion 120 and the tubing 110 and/or rigid connector 118. In the depicted example, the sealing portion 130 is formed from a resilient material that can accommodate variations between the mating geometry of the tubing portion 120 and the tubing 110 and/or the rigid connector 118. The sealing portion 130 can be formed from a material with a relatively low elastic modulus. In some embodiments, the elastic modulus of the sealing portion 130 is lower than the elastic modulus of the connector 100 and/or the rigid connector 118. The sealing portion 130 can be formed from soft, spring-like materials, such as elastomers. Optionally, the sealing portion 130 can be formed from a common material to simplify manufacturing and testing of fluid joints. In some embodiments, the sealing portion 130 can be formed from the same material as the tubing 110 (e.g. the sealing portion 130 and the tubing 110 can both be formed from PVC). Advantageously, by forming the sealing portion 130 from the same material as the tubing 110, the tubing 110 can bond or engage with the sealing portion more effectively compared to traditional bonding.

In some embodiments, the sealing portion 130 is disposed within the connector lumen 126 to fill any gaps between the inner surface 134 and the tubing 110 and/or the rigid connector 118. As illustrated the inner surface 134 can define a sealing portion lumen. Further, the sealing portion 130 can further conform or otherwise resiliently engage any gaps between the outer surface 132 and the inner surface 117 of the connector lumen 126.

Optionally, the sealing portion 130 can have a generally cylindrical shape. In some embodiments, the sealing portion 130 can have a generally circular profile or a non-circular profile. The sealing portion 130 can extend at a distal end 136 toward the coupling portion 122. The proximal end 138 can extend partially into the connector lumen 126. In some embodiments, the sealing portion 130 is embedded into or otherwise coupled with the connector lumen 126. Optionally, the sealing portion 130 can be embedded into or otherwise coupled with the tubing 110 and/or the rigid connector 118. In some applications, the sealing portion 130 is overmolded or co-molded with the connector 100, the tubing 110, and/or the rigid connector 118.

In some embodiments, the sealing portion 130 may be circumferentially disposed around the tubing 110 and/or the rigid connector 118. In some embodiments, the sealing portion 130 can be partially circumferentially disposed around the tubing 110 and/or the rigid connector 118. Optionally, the sealing portion 130 may include alternating portions of resilient and stiff (e.g. low elastic modulus material and high elastic modulus material) to provide a range of clamping force as a function of axial displacement.

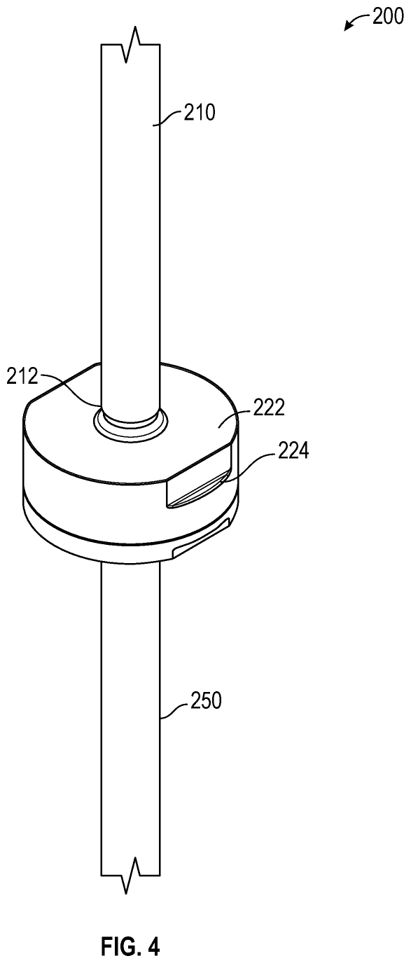

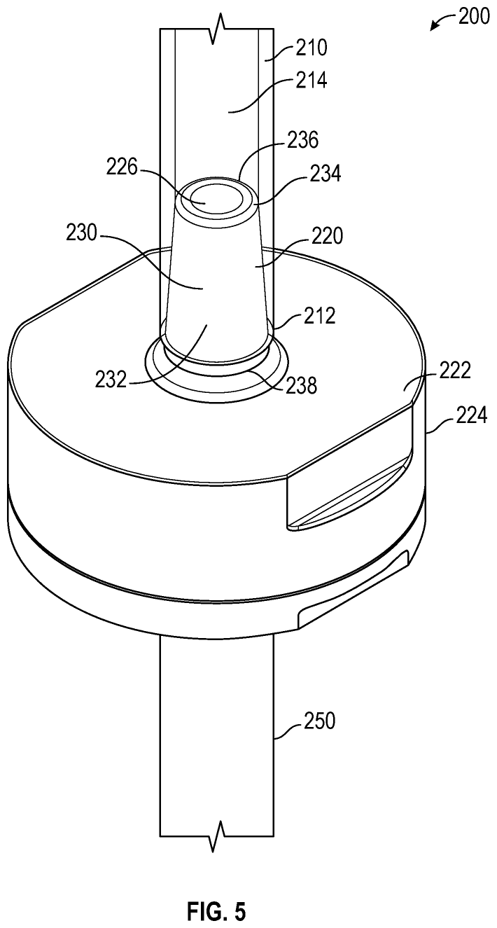

FIG. 4 illustrates a perspective view of a connector 200, according to certain aspects of the present disclosure. FIG. 5 illustrates a perspective view of a connector 200 with the tubing shown in hidden lines. In the depicted example, the connector 200 includes features that are similar to the features of the connector 100. Similar features may be referred to with similar reference numerals. As illustrated, the tubing 210 can be terminated with a connector 200 to allow for the connection and/or disconnection of the tubing 210 to allow flow between the tubing 110, the tubing 250, and/or other components of the IV set.

As illustrated, the tubing 210 can be coupled with, or engage with a tubing portion 220 of the connector 200. The tubing portion 220 can be in fluid communication with a tubing lumen 214 of the tubing 210 to allow fluid to pass through the connector 200. In some embodiments, an outer surface of the tubing portion 220 can engage or couple with the tubing lumen 214. In some embodiments, portions of the tubing 210 can be inserted over the tubing portion 220 of the connector 200 to engage with, or otherwise couple the connector 200. As illustrated, the tubing end 212 can be advanced toward the coupling portion 222 of the connector 200.

Fluid can flow from through the connector 200 via a coupling portion 222 extending from the tubing portion 220. The connector lumen 226 can be in fluid communication with the coupling portion 222. As illustrated, the coupling portion 222 can engage or couple with a mating coupling portion to permit flow through the connector to another portion of tubing 250.

Optionally, the coupling portion 122 can include features 124 disposed on the surface of the connector 200 to allow a clinician to more easily handle or manipulate the connector 200. The raised features 224 can allow the connector 200 to be engaged with mating portions of the connector 200.

In the depicted example, the connector 200 can include a sealing portion 230 to sealingly engage with the tubing 210 to securely couple the tubing 210 to the connector 200. Similar to sealing portion 130, the sealing portion 230 can be formed from a material with a relatively low elastic modulus. In some embodiments, the elastic modulus of the sealing portion 230 is lower than the elastic modulus of the connector 200.

In some embodiments, the sealing portion 230 is disposed around the outer surface of the tubing portion 220 to fill any gaps between the outer surface 234 and the tubing 210. Further, the sealing portion 230 can further conform or otherwise resiliently engage any gaps between the inner surface 232 and the outer surface of the tubing portion 220.

In some embodiments, the sealing portion 230 is embedded into or otherwise coupled with an outer surface of the tubing portion 220. In some applications, the sealing portion 230 is overmolded or co-molded with the tubing portion 220.

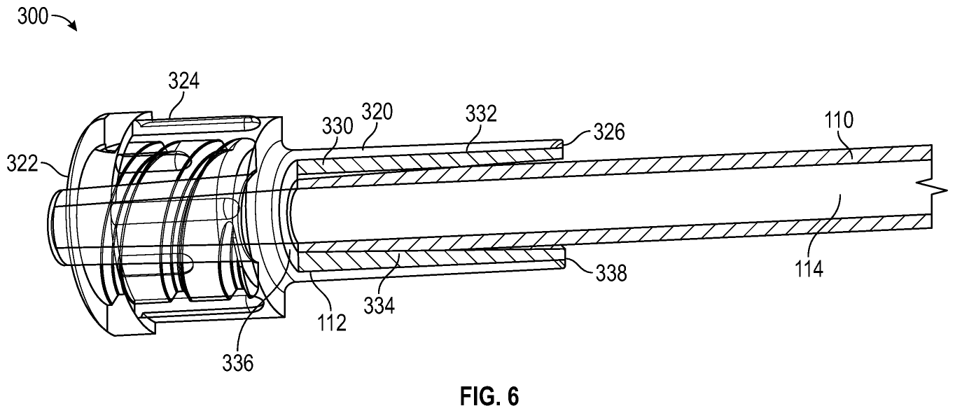

FIG. 6 illustrates a perspective view of a connector 300, according to certain aspects of the present disclosure. In the depicted example, the connector 300 includes features that are similar to the features of the connector 100. Similar features may be referred to with similar reference numerals. In the directed example, the tubing 110 without a rigid connector can be terminated with a connector 300 to allow for the connection and/or disconnection of the tubing 110 to allow fluid flow between the tubing 110 and components of the IV set.

As described herein, the tubing 110 can be coupled with, or engage with a tubing portion 320 of the connector 300. As illustrated, the tubing end 112 of the tubing 110 can be advanced distally into the connector lumen 236. In some embodiments, the tubing end 112 can be advanced to be adjacent to the coupling portion 322 of the connector 300.

As illustrated, the sealing portion 330 can have a generally cylindrical shape. The sealing portion 330 can extend at a distal end 336 toward the coupling portion 322. The proximal end 138 can extend across the length of the connector lumen 326, increasing the engagement or bond surface between the sealing portion 330 and the tubing 110.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A connector comprising:
a coupling portion configured to engage with a mating connector;
a tubing portion extending from the coupling portion, the tubing portion comprising a tubing portion elastic modulus and the tubing portion defining a lumen in fluid communication with the coupling portion, wherein the tubing portion is configured to engage with a tubing; and
a sealing portion coupled to the tubing portion, the sealing portion comprising a sealing portion elastic modulus, wherein the sealing portion elastic modulus is lower than the tubing portion elastic modulus, and the sealing portion is configured to sealingly engage against the tubing.

2. The connector of claim 1, wherein the sealing portion comprises a same material as the tubing.

3. The connector of claim 1, wherein the tubing portion is configured to receive the tubing within the lumen.

4. The connector of claim 3, wherein the sealing portion is coupled to an inner surface of the lumen.

5. The connector of claim 4, wherein the sealing portion is comolded with the tubing portion.

6. The connector of claim 1, wherein the tubing portion is configured to receive the tubing around the tubing portion.

7. The connector of claim 6, wherein the sealing portion is coupled to an outer surface of the tubing portion.

8. The connector of claim 7, wherein the sealing portion is overmolded with the tubing portion.

9. A method comprising:
advancing a tubing toward a connector;
engaging the tubing with a tubing portion of the connector; and
sealingly engaging the tubing with the tubing portion of the connector via a sealing portion coupled to the tubing portion, wherein a sealing portion elastic modulus is lower than a tubing portion elastic modulus.

10. The method of claim 9, further comprising:
filling voids defined between the tubing and the tubing portion of the connector via the sealing portion.

11. The method of claim 9, further comprising:
advancing the tubing into a lumen defined by the tubing portion of the connector.

12. The method of claim 9, further comprising:
advancing a tubing lumen of the tubing over the tubing portion of the connector.

13. The method of claim 9, further comprising:
comolding the sealing portion with the tubing portion of the connector.

14. The method of claim 9, further comprising:
overmolding the sealing portion with the tubing portion of the connector.

15. A connector assembly comprising:
a connector comprising:
a coupling portion configured to engage with a mating connector;
a tubing portion extending from the coupling portion, the tubing portion comprising a tubing portion elastic modulus and the tubing portion defining a connector lumen in fluid communication with the coupling portion; and
a sealing portion coupled to the tubing portion, the sealing portion comprising a sealing portion elastic modulus, wherein the sealing portion elastic modulus is lower than the tubing portion elastic modulus; and
a tubing defining a tubing lumen, wherein the tubing is sealingly engaged with the sealing portion and the tubing lumen is in fluid communication with the connector lumen.

16. The connector assembly of claim 15, wherein the tubing comprises an integrated connector, wherein the integrated connector is sealingly engaged with the sealing portion of the connector.

17. The connector assembly of claim 16, wherein the integrated connector comprises an integrated connector elastic modulus and the integrated connector elastic modulus is greater than the sealing portion elastic modulus.

18. The connector assembly of claim 16, wherein the integrated connector comprises a tapered geometry.

19. The connector assembly of claim 15, wherein the sealing portion comprises an elastomer.

20. The connector assembly of claim 15, wherein the tubing portion is configured to receive the tubing within the connector lumen.
